(19) United States
(12) Patent Application Publication
(10) Pub. No.: US 2009/0312222 A1
(43) Pub. Date: Dec. 17, 2009

(54) Laundry Composition

(76) Inventors: Paul Ferguson, Wirral (GB); Robert Alan Hunter, Wirral (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/085,615

(22) PCT Filed: Nov. 6, 2006
(86) PCT No.: PCT/EP2006/010715

§ 371 Date: May 28, 2008

(30) Foreign Application Priority Data
Dec. 2, 2005 (GB) 0524665.7

(51) Int. Cl. C11D 3/50; A61K 8/02; A61Q 13/00

(52) U.S. Cl. 510/337; 512/4; 510/276

(57) ABSTRACT

Polymer particles comprising a perfume are delivered to fabric during laundering and give long lasting adherence of the perfume to fabric during laundering. The particle comprises a polymer and is prepared using miniemulsion polymerisation. A method of depositing a perfume onto a substrate by means of such a composition is also disclosed.

### TECHNICAL FIELD

The present invention relates to polymer particles comprising a perfume and a nonionic deposition aid and their uses, such as delivery of the perfume to fabric during laundering. Laundry treatment compositions containing particles according to the invention, provide deposition efficiency benefits during washing.

### BACKGROUND OF THE INVENTION

The deposition of a perfume onto a substrate, such as a fabric, is a well known method of imparting perfume properties to the substrate. In laundry applications deposition of a perfume is used, for example, during fabric treatment processes such as fabric conditioning. Methods of deposition are diverse and include deposition during the wash or rinse stages of the laundry process or direct deposition before or after the wash, such as by spraying or rubbing or by use of impregnated sheets during tumble drying or water additives during steam ironing.

The perfume is often incorporated into a carrier or delivery system. Carrier systems for perfumes are typically based on encapsulation or entrapment within a matrix. The perfume may simply be emulsified but problems with poor retention or stability exist and deposition onto a substrate, such as fabric, is often inefficient. Diffusion of the perfume into a carrier suffers from complex preparation including time of diffusion; poor retention of the perfume in the matrix and subsequent poor substrate deposition are also common problems.

After deposition onto a surface, a problem exists in that longevity of adherence to that surface of the perfume, in a surfactant containing environment, is inherently poor because surfactants are characteristically very efficient at combining with perfumes. This results in a perfume which has been deposited onto a fabric being washed off during a main wash, or in the perfume being leached from its carrier in the wash liquor thus becoming unavailable for deposition onto the fabric. Protection of the perfume is, therefore, required before and after it has been deposited onto a surface. In the context of laundry products, the need for protection from surfactants of perfumes promotes the need for new protection systems. By protection benefit as used herein is meant protection of the perfume from the action of surfactants during a wash process, for example as suggested above. Thus the protection of perfumes within a formulation in an aqueous environment and longevity of the perfume deposited onto a fabric are both desirable goals.

Protection from the action of surfactants and longevity of deposition on a substrate are particular needs for perfumes as the volatile nature and low molecular weight of perfume components make them susceptible to diffusing out of carrier systems during laundering and evaporating quickly from substrates after deposition.

### PRIOR ART

Our co-pending patent application, PCT/EP2005/004779, unpublished at the filing date of this application, is directed towards a process, which uses miniemulsion polymerisation, for the preparation of polysaccharide/polymer conjugate particles containing a lubricant. Certain particles produced by the process and uses thereof are also disclosed. The particles facilitate deposition of the lubricant to fabric during the main wash part of a laundering process.

Our co-pending patent applications, GB 0513803.7 and GB 051805.2, both unpublished at the filing date of this application, are directed towards miniemulsion polymer particles (with and without a shell respectively) comprising a benefit agent, preferably a sugar polyester, which may be delivered to fabric during laundering. These particles give long lasting adherence of the benefit agent to fabric during laundering.

We have now surprisingly found that a perfume can be efficiently deposited onto a fabric if a carrier system based on a colloidal particle comprising a polymer, a perfume and a nonionic deposition aid, such as locust bean gum is employed. The perfume is not post-absorbed/adsorbed into/onto the particle, but instead is incorporated into the particle as the particle is formed. The carrier system further provides protection and longevity of adherence to the perfume.

### DEFINITION OF THE INVENTION

A first aspect of the invention provides a polymer particle comprising a perfume and a polymer comprising monomer units which are derived from monomers selected from the group consisting of:
a) monomers with a solubility in water of less than 0.1 g/l, and/or
b) monomers with a solubility in water of from 0.1 to 30 g/l, and
c) optionally, monomers with a solubility in water of greater than 30 g/l, and/or
d) optionally, cross linkers;

and wherein the particle further comprises a nonionic deposition aid.

A second aspect of the invention provides a process for the preparation of the particles according to the first aspect, which comprises miniemulsion polymerisation of monomers.

A third aspect of the invention provides a laundry treatment composition comprising the particles of the first aspect. Use of this laundry treatment composition to provide a perfume deposition benefit to fabric is also provided.

In a fourth aspect, the invention provides the use of a particle of the first aspect to provide a perfume deposition benefit during a laundry process.

In a further aspect, the invention provides the use of a particle of the first aspect in the manufacture of a laundry treatment composition to provide a perfume deposition benefit during the laundry process.

In a still further aspect, the invention provides an aqueous wash medium comprising from 0.05 to 1 gram per litre of a particle according to the first aspect of the invention.

### DETAILED DESCRIPTION OF THE INVENTION

The present invention is directed towards polymer particles, comprising a core, wherein the core comprises perfume and a polymer, which comprises monomer units; the particles further comprise a nonionic deposition aid. The polymer particles of the invention are usually approximately spherical and of typical colloidal dimensions. Particle diameters may range from about 30 to 500 nm (The Encyclopaedia of Polymer Science and Engineering, Second Edition, Volume 8, Page 647, John Wiley and Sons Inc. (1987)).

Where the particles of the invention are described herein as latex particles, the term “latex” or “latex particle” is defined as a stable colloidal dispersion of a polymeric substance in an aqueous medium.

The polymer particles of the invention can comprise a wide range of monomeric units. By “monomer units” as used herein is meant the monomeric units of the polymer chain, thus references to “a polymer particle comprising insoluble monomer units” as used herein means that the polymer particles is derived from insoluble monomers, and so forth.

The monomer units are derived from monomers which are suitable for free radical polymerisation. Therefore, preferably the monomer contains at least one ethylenically unsaturated group capable of undergoing addition polymerisation.

The monomers may be selected according to their solubilities such that the polymer comprises monomer units which are derived from monomers selected from the group consisting of:
a) monomers with a solubility in water of less than 0.1 g/l, and/or
b) monomers with a solubility in water of from 0.1 to 30 g/l, and
c) optionally, monomers with a solubility in water of greater than 30 g/l, and/or
d) optionally, cross linkers.

By insoluble as used herein in reference to monomers, is meant that the material is soluble in water (distilled or equivalent) at a concentration of less than 0.1 g/litre, at 25° C., i.e. monomers of type (a) above.

By low solubility as used herein in reference to monomers, is meant that the material is soluble in water (distilled or equivalent) at a concentration in the range of from 0.1 to 30 g/litre, at 25° C., i.e. monomers of type (b) above.

By high solubility as used herein in reference to monomers, is meant that the material is soluble in water (distilled or equivalent) at a concentration of greater than 30 g/litre, at 25° C., i.e. monomers of type (c) above.

The particle of the invention may further comprise a shell, which comprises monomer units selected from (b), and optionally (c) and/or optionally (d).

Monomers

The polymer comprises monomer units which are derived from monomers that are capable of undergoing free radical polymerisation. Suitable classes of such monomers are given in the group consisting of olefins, ethylene, vinylaromatic monomers, esters of vinyl alcohol with mono- and di-carboxylic acids, esters of α,β-monoethylenically unsaturated mono- and dicarboxylic acids with alcohols, nitrites of α,β-monoethylenically unsaturated carboxylic acids, conjugated dienes, α,β-monoethylenically unsaturated monocarboxylic and dicarboxylic acids and their amides, methacrylic acid and its esters with alcohols and diols, acrylic acid and its esters with alcohols and diols, dimethyl or di-n-butyl maleate, and vinyl-sulfonic acid and its water-soluble salts, and mixtures thereof. The polymer particle may comprise mixtures of monomer units.

The polymer particle may optionally comprise monomers which are cross-linkers. Such crosslinkers may have at least two non-conjugated ethylenically unsaturated double bonds. Examples are alkylene glycol diacrylates and dimethacrylates. A further type of suitable cross-linking monomers are those that are conjugated, such as divinyl benzene. If present, these monomers constitute from 0.1 to 10% by weight, based on the total amount of monomers to be polymerised.

The monomers a), b), c) and d), as defined above, are preferably selected from the following:
a) vinyl octate; Vinyl decanote; vinyl laurate; vinyl stearate; esters of acrylic, methacrylic, maleic, fumaric or itaconic acid with decyl, dodecyl, tetadecyl, hexadecyl and octadecyl alcohol,
b) styrene; a-methylstyrene; o-chlorostyrene; vinyl acetate; vinyl propionate; vinyl n-butyrate; esters of acrylic, methacrylic, maleic, fumaric or itaconic acid with methyl, ethyl, n-butyl, isobutyl, n-hexyl and 2-ethylhexyl alcohol; 1,3-butadiene; 2,3 dimethyl butadiene; and isoprene,
c) acrylic acid, methacrylic acid, maleic acid, fumaric acid, itaconic acid, poly (alkylene oxide) monoacrylates and monomethacrylates, N-vinyl-pyrrolidone, methacrylic and acrylic acid, 2-hydroxyethyl acrylates and methacrylates, glycerol acrylates and methacrylates, poly(ethylene glycol) methacrylates and acrylates, n-vinyl pyrrolidone, acryloyl morpholine, vinyl formamide, n-vinyl acetamide and vinyl caprolactone, acrylonitrile (71 g/l), acrylamide, and methacrylamide at levels of less than 10% by weight of the monomer unit content of the particle; 2-(dimethylamino) ethyl methacrylate, 2-(diethylamino) ethyl methacrylate, 2-(tert-butylamino) ethyl methacrylate, 2- aminoethyl methacrylate, 2-(2-oxo-1-imidazolidinyl) ethyl methacrylate, vinyl pyridine, vinyl carbazole, vinyl imidazole, vinyl aniline, and their cationic forms after treatment with alkyl halides;
d) vinyltoluenes, divinyl benzene, ethylene glycol diacrylate, 1,2-propylene glycol diacrylate, 1,3-propylene glycol diacrylate, 1,3-butylene glycol diacrylate, 1,4-butylene glycol diacrylates, ethylene glycol dimethacrylate, 1,2-propylene glycol dimethacrylate, 1,3-propylene glycol dimethacrylate, 1,3-butylene glycol dimethacrylate, 1,4-butylene glycol dimethacrylate, divinylbenzene, vinyl methacrylate, vinyl acrylate, allyl methacrylate, allyl acrylate, diallyl maleate, diallyl fumarate, methylenebisacrylamide, cyclopentadienyl acrylate, and triallyl cyanurate.

The polymer particle may comprise monomer units, which are derived from monomers selected from the group consisting of butyl acrylate, butyl methacrylate, hexyl acrylate, hexyl methacrylate, 2-ethylhexyl acrylate, 2-ethylhexyl methacrylate, dodecyl acrylate, dodecyl methacrylate, octadecyl acrylate, octadecyl methacrylate, styrene, vinyl acetate and divinyl benzene, or mixtures thereof.

Particles of the invention can be optionally comprise monomer units which are derived from monomers of solubility of greater than about 30 g/litre, preferably greater than 35 g/litre, for example 40 to 45 g/litre in water (distilled or equivalent) at 25° C. Such monomers may be utilised in a monomer mixture at levels of up to 10% based on weight of monomers used.

The surface of the particle (with or without the shell) may comprise hydrophilic or hydrophobic moieties, the nature of which are determined by the choice of the monomer used to produce the particle. No chemical modification or functionalisation of these moieties at the surface or core of the particle so as to alter their hydrophobic or hydrophilic nature after the polymerisation has occurred is performed.

The Deposition Aid

The polymer particle of the invention comprises a deposition aid, which is nonionic.

Preferably, the deposition aid is a polysaccharide. The polysaccharide preferably has a β-1,4-linked backbone.

Preferably the polysaccharide is a cellulose, a cellulose derivative, or another β-1,4-linked polysaccharide having an affinity for cellulose, such as polymannan, polyglucan, polyglucomannan, polyxyloglucan and polygalactomannan or a mixture thereof. More preferably, the polysaccharide is selected from the group consisting of polyxyloglucan and polygalactomannan. For example, preferred polysaccharides are locust bean gum, tamarind xyloglucan, guar gum or mixtures thereof. Most preferably, the deposition aid is locust bean gum.

The polysaccharide acts as a delivery aid/deposition agent for the particle.

Preferably, the polysaccharide backbone has only β-1,4 linkages. Optionally, the polysaccharide has linkages in addition to the β-1,4 linkages, such as β-1,3 linkages. Thus, optionally some other linkages are present. Polysaccharide backbones which include some material which is not a saccharide ring are also within the ambit of the present invention (whether terminal or within the polysaccharide chain).

The polysaccharide may be straight or branched. Many naturally occurring polysaccharides have at least some degree of branching, or at any rate at least some saccharide rings are in the form of pendant side groups (which are therefore not in themselves counted in determining the degree of substitution) on a main polysaccharide backbone.

Preferably, the polysaccharide is present at levels of between 0.1% to 10% w/w by weight of the total amount of the particle.

The deposition aid, which is preferably a polysaccharide, is attached to the polymer particle (with or without shell). Attachment may be by means of a covalent bond, entanglement or strong adsorption, preferably by a covalent bond or entanglement and most preferably by means of a covalent bond. By entanglement as used herein is meant that the deposition aid is adsorbed onto the particle as the polymerisation proceeds and the particle grows in size, part of the adsorbed polysaccharide becomes buried within the interior of the particle. Hence at the end of the polymerisation, part of the polysaccharide is entrapped and bound in the polymer matrix of the particle, whilst the remainder is free to extend into the aqueous phase.

By strong adsorption as used herein is meant strong adsorption of the polysaccharide to the surface of the particle; such adsorption can, for example, occur due to hydrogen bonding, Van Der Waals or electrostatic attraction between the polysaccharide chains and the particle.

The deposition aid is thus mainly attached to the particle surface and is not, to any significant extent, distributed throughout the internal bulk of the particle. This is distinct from graft copolymers in which e.g. a polysaccharide may be grafted along the length of a polymer chain. A particle which is formed from a graft copolymer would, therefore, contain polysaccharide throughout the internal bulk of the particle as well as on the particle surface and the present invention is not intended to cover such a particle. Thus the particle which is produced when using a polysaccharide as the deposition aid according to the process of the invention can be thought of as a “hairy particle”, which is different from a graft copolymer. This feature of the invention provides significant cost reduction opportunities for the manufacturer as much less polysaccharide is required to achieve the same level of activity as systems which utilise polysaccharide copolymers.

Other types of particle surface morphology may be produced when a deposition aid is attached to the particle of the invention. For example, where a polysaccharide attaches to the particle surface in multiple places, loops may result, or the deposition aid may be in the form of a swollen cationic polymer layer at the particle surface.

Optional Shell

The particles of the invention may optionally comprise a shell which is situated between the core and the deposition aid. The particle thus has core-shell morphology. Like the core, the shell comprises monomer units which are derived from monomers that are capable of undergoing free radical polymerisation. The shell comprises monomer units selected from b), and optionally c) and/or optionally d) as described above. The monomer units of the core may be the same as or different from the monomer units of the shell and both the core and the shell may comprise mixtures of monomer units. Like the core, the shell may optionally comprise monomers which are cross-linkers as described above. The shell preferably covers 50 to 100% of the surface area of the core, most preferably 95 to 100%.

The ratio of core to shell by weight may be from 1:1 to 100:1, preferably 2:1 to 20:1.

The polymer and perfume must be compatible, i.e. phase separation should not occur when the polymerisation of the monomer (which is mixed with the perfume) is carried out during the process of the invention. The person skilled in the art will be able to determine which are compatible by performing simple tests, for example, a simple test would be to carry out the polymerisation to form a latex, let a sample of the newly formed latex dry into a thin film and look for visual evidence of phase separation—if the film is clear, they are compatible, if opaque they are incompatible. Where an incompatibility exists, dilution of an incompatible polymer with a compatible one may improve its compatibility.

Perfumes

The polymer particle of the invention comprises a perfume. The perfume is present in an amount of from 1 to 50% by total weight of the particle, preferably from 10 to 50% by total weight of the particle.

The perfume suitably has a molecular weight of from 50 to 500.

The perfume suitably has a boiling point of from 30 to 500 degrees Centrigrade.

The perfume is not post-absorbed/adsorbed into/onto the particle, but instead is incorporated into the particle as the particle is formed.

The Polymer Particle

The polymer particle may be used in the treatment of fabric. The treatment provides a perfume benefit.

Alternatively, the polymer particle may be incorporated into a laundry treatment composition and used in the treatment of fabric.

The particle may be used in the manufacture of a laundry treatment composition to provide a perfume benefit during a laundry process.

Processes for Preparation

Any suitable process may be employed for the preparation of the particles of the invention. A suitable process for the preparation of the polymer particle of the invention is one that comprises miniemulsion.

Preferred processes are the so-called “hybrid” or “artificial” routes as detailed in “Miniemulsion Polymerisation”, F. Joseph Schork, Yingwu Luo, Wilfried Smuldes, James P. Russian, Alessandro Butte and Kevin Fontenot, Adv. Polymer Sci (2005) 175: 129-255, and “Emulsion Polymerisation and Emulsion Polymers” Wiley, 1^{st} Edn., 1997, Ed Peter Lovell and Mohamed S El-Aasser p 712, respectively.

Miniemulsion polymerisation is well known in the art and the term “miniemulsion polymerisation” as used herein means the same as the term known in the art. Numerous scientific reviews of miniemulsion techniques have been published:
1) El Aasser, M. S., Miller, C. M., “Preparation of latexes using miniemulsions”, In: Asua, J. M., editor. Polymeric dispersions. Principles and applications. Dordrecht: Kluwer, p. 109-126 (1997)
2) Sudol, E. D., El Aasser, M. S., “Miniemulsion polymerisation”, In: Lovell, P. A., El Aasser, M. S., editors. Emulsion polymerisation and emulsion polymers. Chichester: Wiley, p. 699-722 (1997)
3) Asua, J. M., Prog. Polym. Sci., 27, 1283-1346 (2002)

Miniemulsions generally lie in between macroemulsion and microemulsions in terms of droplet size and emulsion stability. Miniemulsion droplets typically range in size from 50 to 500 nm.

The preferred “hybrid” process comprises the steps of:
(a) preparation of a miniemulsion (comprising monomers, a perfume and a cosurfactant), and
(b) polymerisation of the miniemulsion of step (a) to form miniemulsion core particles, and attachment of a deposition aid around the core particles.

A core-shell particle may be prepared by using the hybrid process as follows:
(a) preparation of a miniemulsion (comprising monomers, a perfume and a cosurfactant), and
(b) polymerisation of the miniemulsion of step (a) to form miniemulsion core particles, and
(c) addition of shell monomers and initiator such that polymerisation occurs to form a shell around the core particles of step (b) and attachment of a deposition aid around the particles.

Step (a) may consist of the following sub-steps:
(i) mixing monomers and perfume with a cosurfactant to form a mixture (y),
(ii) dissolving at least one surfactant in water to form a solution (z),
(iii) combining (y) and (z) and subjecting to high shear to form a miniemulsion.

Step (iii) may consist of the following sub-steps:
(1) (y) and (z) are combined and subjected to high shear to form an emulsion,
(2) the emulsion of step (1) is then subjected to further shear (such as sonication or other suitable high pressure homogeniser such as a Microfluidiser or a Manton Gaulin homogeniser) to form a miniemulsion.

By mixed is meant mixed or dissolved, depending on the physical state of the perfume.

Preferably, an initiator is added to the miniemulsion of step (iii) or step (2) such that polymerisation of the monomers proceeds. Alternatively, the initiator can be added during step (a), for example if an initiator that is soluble in the monomer is used, or in step (b), for example if the initiator is water soluble.

Alternatively, the surfactant of step (ii) may be a reactive surfactant, by which is meant that it comprises groups that may participate in the polymerisation and a hydrophilic group. In this case it will be combined with the monomer in step (i).

The preferred “artificial.” process comprises the steps of:
(a) preparation of a miniemulsion (comprising a polymer, a perfume, a cosurfactant and a water insoluble volatile solvent),
(b) removal of the volatile solvent, and
(c) addition of shell monomers and subsequent polymerisation to form a shell around the core particles of step (b), wherein attachment of a deposition aid to the particles occurs in step (c).

Step (a) may consist of the following sub-steps:
(i) dissolving polymer and a perfume with a cosurfactant in a water insoluble volatile solvent to form a mixture solution (m),
(ii) dissolving at least one surfactant in water to form a mixture (n), and
(iii) combining (m) and (n) and subjecting to high shear to form a miniemulsion.

A suitable solvent is dicholormethane (DCM).

The resulting polymer particles preferably have a particle size of less than 1 micron, more preferably of less than 500 nm.

High shear as used herein is applied using any suitable apparatus such as an ultrasound sonicator, microfluidizer or homogenizer. High shear as used in step (iv) above is defined as shear of sufficiently high intensity that the emulsion of step (iii) above is reduced in particle size to sub-micron dimensions, preferably under 500 nm. Suitably, the emulsion of step (iv) is formed using a high shear homogeniser at 10,000 to 24,000 rpm for approximately 30 seconds to 5 minutes and then sonified using a probe ultrasound sonicator (at maximum power output) for 10 minutes to generate the miniemulsion. A suitable homogeniser is a Manton Gaulin homogeniser or any other make of high shear homogeniser such as an Ultra Turrax.

Cosurfactants, Initiators and Chain Transfer Agents

The monomer (hybrid process) or polymer solution (artificial process) is mixed with a cosurfactant. Suitable cosurfactants for use in the present invention include hexadecane, cetyl alcohol, lauroyl peroxide, n-dodecyl mercaptan, dodecyl methacrylate, stearyl methacrylate, polystyrene and polymethyl methacrylate. The preferred cosurfactant comprises hexadecane.

Initiators and chain transfer agents may also be present. Those skilled in the art will recognise that a chemical initiator will generally be required but that there are instances in which alternative forms of initiation will be possible, e.g. ultrasonic initiation or initiation by irradiation.

The initiator is preferably a chemical or chemicals capable of forming free radicals. Typically, free radicals can be formed either by homolytic scission (i.e. homolysis) of a single bond or by single electron transfer to or from an ion or molecule (e.g. redox reactions).

Suitably, in context of the invention, homolysis may be achieved by the application of heat (typically in the range of from 50 to 100° C.). Some examples of suitable initiators in this class are those possessing peroxide (—O—O—) or azo (—N═N—) groups, such as benzoyl peroxide, t-butyl peroxide, hydrogen peroxide, azobisisobutyronitrile and ammonium persulphate. Homolysis may also be achieved by the action of radiation (usually ultraviolet), in which case it is termed photolysis. Examples are the dissociation of 2,2′-azobis (2-cyanopropane) and the formation of free radicals from benzophenone and benzoin.

Redox reactions can also be used to generate free radicals. In this case an oxidising agent is paired with a reducing agent which then undergo a redox reaction. Some examples of appropriate pairs in the context of the invention are ammonium persulphate/sodium metabisulphite, cumyl hydroperoxide/ferrous ion and hydrogen peroxide/ascorbic acid.

Preferred initiators are selected from the following:

Homolytic: benzoyl peroxide, t-butyl peroxide, hydrogen peroxide, azobisisobutyronitrile, ammonium persulphate, 2,2′-azobis (cyanopropane), benzophenone, benzoin, Redox: ammonium persulphate/sodium metabisulphite mixture, cumyl hydroperoxide/ferrous ion mixture and/or hydrogen peroxide/ascorbic acid mixture. Preferred initiators are ammonium persulphate and hydrogen peroxide/ascorbic acid mixture. The preferred level of initiator is in the range of from 0.1 to 5.0% w/w by weight of monomer, more preferably, the level is in the range of from 1.0 to 3.0% w/w by weight of monomer.

Chain transfer agents can optionally be used to reduce the degree of polymerisation and hence the final molecular weight of the polymer. A chain transfer agent contains very labile hydrogen atoms that are easily abstracted by a propagating polymer chain. This terminates the polymerisation of the growing polymer, but generates a new reactive site on the chain transfer agent that can then proceed to initiate further polymerisation of the remaining monomer. Chain transfer agents in the context of the invention typically contain thiol (mercaptan) functionality and can be represented by the general chemical formula RS—H, such as n-dodecyl mercaptan and 2-mercaptoethanol. Preferred chain transfer agents are monothioglycerol and n-dodecyl mercaptan, used at levels of, preferably from 0 to 5% w/w based on the weight of the monomer and more preferably at a level of 0.25% w/w based on the weight of the monomer.

Laundry Treatment Compositions

The polymer particles of the invention may be incorporated into laundry compositions.

The polymer particles are typically included in said compositions at levels of from 0.001% to 10%, preferably from 0.005% to 5%, most preferably from 0.01% to 3% by weight of the total composition.

The active ingredient in the compositions is preferably a surface active agent or a fabric conditioning agent. More than one active ingredient may be included. For some applications a mixture of active ingredients may be used.

The compositions of the invention may be in any physical form e.g. a solid such as a powder or granules, a tablet, a solid bar, a paste, gel or liquid, especially, an aqueous based liquid. In particular the compositions may be used in laundry compositions, especially in liquid, powder or tablet laundry composition.

The compositions of the present invention are preferably laundry compositions, especially main wash (fabric washing) compositions or rinse-added softening compositions. The main wash compositions may include a fabric softening agent and the rinse-added fabric softening compositions may include surface-active compounds, particularly non-ionic surface-active compounds.

The detergent compositions of the invention may contain a surface-active compound (surfactant) which may be chosen from soap and non-soap anionic, cationic, non-ionic, amphoteric and zwitterionic surface-active compounds and mixtures thereof. Many suitable surface-active compounds are available and are fully described in the literature, for example, in “Surface-Active Agents and Detergents”, Volumes I and II, by Schwartz, Perry and Berch.

The preferred detergent-active compounds that can be used are soaps and synthetic non-soap anionic, and non-ionic compounds.

The compositions of the invention may contain linear alkylbenzene sulphonate, particularly linear alkylbenzene sulphonates having an alkyl chain length of from C8 to C15. It is preferred if the level of linear alkylbenzene sulphonate is from 0 wt % to 30 wt %, more preferably from 1 wt % to 25 wt %, most preferably from 2 wt % to 15 wt %, by weight of the total composition.

The compositions of the invention may contain other anionic surfactants in amounts additional to the percentages quoted above. Suitable anionic surfactants are well-known to those skilled in the art. Examples include primary and secondary alkyl sulphates, particularly C8 to C15 primary alkyl sulphates; alkyl ether sulphates; olefin sulphonates; alkyl xylene sulphonates; dialkyl sulphosuccinates; and fatty acid ester sulphonates. Sodium salts are generally preferred.

The compositions of the invention may also contain non-ionic surfactant. Nonionic surfactants that may be used include the primary and secondary alcohol ethoxylates, especially the C8 to C20 aliphatic alcohols ethoxylated with an average of from 1 to 20 moles of ethylene oxide per mole of alcohol, and more especially the C10 to C15 primary and secondary aliphatic alcohols ethoxylated with an average of from 1 to 10 moles of ethylene oxide per mole of alcohol. Non-ethoxylated nonionic surfactants include alkylpolyglycosides, glycerol monoethers, and polyhydroxyamides (glucamide).

It is preferred if the level of non-ionic surfactant is from 0 wt % to 30 wt %, preferably from 1 wt % to 25 wt %, most preferably from 2 wt % to 15 wt %, by weight of the total composition.

Any conventional fabric conditioning agent may be used in the compositions of the present invention. The conditioning agents may be cationic or non-ionic. If the fabric conditioning compound is to be employed in a main wash detergent composition the compound will typically be non-ionic. For use in the rinse phase, typically they will be cationic. They may for example be used in amounts from 0.5% to 35%, preferably from 1% to 30% more preferably from 3% to 25% by weight of the composition.

Suitable cationic fabric softening compounds are substantially water-insoluble quaternary ammonium materials comprising a single alkyl or alkenyl long chain having an average chain length greater than or equal to C20 or, more preferably, compounds comprising a polar head group and two alkyl or alkenyl chains having an average chain length greater than or equal to C14. Preferably the fabric softening compounds have two long chain alkyl or alkenyl chains each having an average chain length greater than or equal to C16. Most preferably at least 50% of the long chain alkyl or alkenyl groups have a chain length of C18 or above. It is preferred if the long chain alkyl or alkenyl groups of the fabric softening compound are predominantly linear.

Quaternary ammonium compounds having two long-chain aliphatic groups, for example, distearyldimethyl ammonium chloride and di(hardened tallow alkyl) dimethyl ammonium chloride, are widely used in commercially available rinse conditioner compositions. Other examples of these cationic compounds are to be found in “Surfactants Science Series” volume 34 ed. Richmond 1990, volume 37 ed. Rubingh 1991 and volume 53 eds. Cross and Singer 1994, Marcel Dekker Inc. New York”.

Any of the conventional types of such compounds may be used in the compositions of the present invention.

The fabric softening compounds are preferably compounds that provide excellent softening, and are characterised by a chain melting Lβ to Lα transition temperature greater than 250° C., preferably greater than 350° C., most preferably greater than 450° C. This Lβ to Lα transition can be measured by differential scanning calorimetry as defined in “Handbook of Lipid Bilayers”, D Marsh, CRC Press, Boca Raton, Fla., 1990 (pages 137 and 337).

Substantially water-insoluble fabric softening compounds are defined as fabric softening compounds having a solubility of less than 1×10⁻³ wt % in demineralised water at 20° C. Preferably the fabric softening compounds have a solubility of less than 1×10⁻⁴ wt %, more preferably from less than 1×10⁻⁸ to 1×10⁻⁶ wt %.

Especially preferred are cationic fabric softening compounds that are water-insoluble quaternary ammonium materials having two C12-22 alkyl or alkenyl groups connected to the molecule via at least one ester link, preferably two ester links. An especially preferred ester-linked quaternary ammonium material can be represented by the formula:

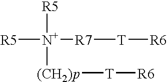

wherein each R5 group is independently selected from C1-4 alkyl or hydroxyalkyl groups or C2-4 alkenyl groups; each R6 group is independently selected from C8-28 alkyl or alkenyl groups; and wherein R7 is a linear or branched alkylene group of 1 to 5 carbon atoms, T is

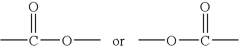

and p is 0 or is an integer from 1 to 5.

Di(tallowoxyloxyethyl) dimethyl ammonium chloride and/or its hardened tallow analogue is an especially preferred compound of this formula.

A second preferred type of quaternary ammonium material can be represented by the formula:

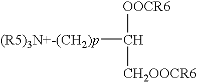

wherein R5, p and R6 are as defined above.

A third preferred type of quaternary ammonium material are those derived from triethanolamine (hereinafter referred to as ‘TEA quats’) as described in for example U.S. Pat. No. 3,915,867 and represented by formula:

(TOCH₂CH₂)₃N+(R9)

wherein T is H or (R8—CO—) where R8 group is independently selected from C8-28 alkyl or alkenyl groups and R9 is C1-4 alkyl or hydroxyalkyl groups or C2-4 alkenyl groups. For example N-methyl-N,N,N-triethanolamine ditallowester or di-hardened-tallowester quaternary ammonium chloride or methosulphate. Examples of commercially available TEA quats include Rewoquat WE18 and Rewoquat WE20, both partially unsaturated (ex. WITCO), Tetranyl AOT-1, fully saturated (ex. KAO) and Stepantex VP 85, fully saturated (ex. Stepan).

It is advantageous if the quaternary ammonium material is biologically biodegradable.

Preferred materials of this class such as 1,2-bis(hardened tallowoyloxy)-3-trimethylammonium propane chloride and their methods of preparation are, for example, described in U.S. Pat. No. 4,137,180 (Lever Brothers Co). Preferably these materials comprise small amounts of the corresponding monoester as described in U.S. Pat. No. 4,137,180, for example, 1-hardened tallowoyloxy-2-hydroxy-3-trimethylammonium propane chloride.

Other useful cationic softening agents are alkyl pyridinium salts and substituted imidazoline species. Also useful are primary, secondary and tertiary amines and the condensation products of fatty acids with alkylpolyamines.

The compositions may alternatively or additionally contain water-soluble cationic fabric softeners, as described in GB 2 039 556B (Unilever).

The compositions may comprise a cationic fabric softening compound and an oil, for example as disclosed in EP-A-0829531.

The compositions may alternatively or additionally contain nonionic fabric softening agents such as lanolin and derivatives thereof.

Lecithins and other phospholipids are also suitable softening compounds.

In fabric softening compositions nonionic stabilising agent may be present. Suitable nonionic stabilising agents may be present such as linear C8 to C22 alcohols alkoxylated with 10 to 20 moles of alkylene oxide, C10 to C20 alcohols, or mixtures thereof. Other stabilising agents include the deflocculating polymers as described in EP 0415698A2 and EP 0458599B1.

Advantageously the nonionic stabilising agent is a linear C8 to C22 alcohol alkoxylated with 10 to 20 moles of alkylene oxide. Preferably, the level of nonionic stabiliser is within the range from 0.1 to 10% by weight, more preferably from 0.5 to 5% by weight, most preferably from 1 to 4% by weight. The mole ratio of the quaternary ammonium compound and/or other cationic softening agent to the nonionic stabilising agent is suitably within the range from 40:1 to about 1:1, preferably within the range from 18:1 to about 3:1.

The composition can also contain fatty acids, for example C8 to C24 alkyl or alkenyl monocarboxylic acids or polymers thereof. Preferably saturated fatty acids are used, in particular, hardened tallow C16 to C18 fatty acids. Preferably the fatty acid is non-saponified, more preferably the fatty acid is free, for example oleic acid, lauric acid or tallow fatty acid. The level of fatty acid material is preferably more than 0.1% by weight, more preferably more than 0.2% by weight. Concentrated compositions may comprise from 0.5 to 20% by weight of fatty acid, more preferably 1% to 10% by weight. The weight ratio of quaternary ammonium material or other cationic softening agent to fatty acid material is preferably from 10:1 to 1:10.

It is also possible to include certain mono-alkyl cationic surfactants which can be used in main-wash compositions for fabrics. Cationic surfactants that may be used include quaternary ammonium salts of the general formula R1R2R3R4N+X− wherein the R groups are long or short hydrocarbon chains, typically alkyl, hydroxyalkyl or ethoxylated alkyl groups, and X is a counter-ion (for example, compounds in which R1 is a C8-C22 alkyl group, preferably a C8-C10 or C12-C14 alkyl group, R2 is a methyl group, and R3 and R4, which may be the same or different, are methyl or hydroxyethyl groups); and cationic esters (for example, choline esters).

The choice of surface-active compound (surfactant), and the amount present, will depend on the intended use of the detergent composition. In fabric washing compositions, different surfactant systems may be chosen, as is well known to the skilled formulator, for handwashing products and for products intended for use in different types of washing machine.

The total amount of surfactant present will also depend on the intended end use and may be as high as 60 wt %, for example, in a composition for washing fabrics by hand. In compositions for machine washing of fabrics, an amount of from 5 to 40 wt % is generally appropriate. Typically the compositions will comprise at least 2 wt % surfactant e.g. 2-60%, preferably 15-40% most preferably 25-35%, by weight of the composition.

Detergent compositions suitable for use in most automatic fabric washing machines generally contain anionic non-soap surfactant, or non-ionic surfactant, or combinations of the two in any suitable ratio, optionally together with soap.

The compositions of the invention, when used as main wash fabric washing compositions, will generally also contain one or more detergency builders. The total amount of detergency builder in the compositions will typically range from 5 to 80 wt %, preferably from 10 to 60 wt %, by weight of the compositions.

Inorganic builders that may be present include sodium carbonate, if desired in combination with a crystallisation seed for calcium carbonate, as disclosed in GB 1 437 950 (Unilever); crystalline and amorphous aluminosilicates, for example, zeolites as disclosed in GB 1 473 201 (Henkel), amorphous aluminosilicates as disclosed in GB 1 473 202 (Henkel) and mixed crystalline/amorphous aluminosilicates as disclosed in GB 1 470 250 (Procter & Gamble); and layered silicates as disclosed in EP 164 514B (Hoechst). Inorganic phosphate builders, for example, sodium orthophosphate, pyrophosphate and tripolyphosphate are also suitable for use with this invention.

The compositions of the invention preferably contain an alkali metal, preferably sodium, aluminosilicate builder. Sodium aluminosilicates may generally be incorporated in amounts of from 10 to 70% by weight (anhydrous basis), preferably from 25 to 50 wt %.

The alkali metal aluminosilicate may be either crystalline or amorphous or mixtures thereof, having the general formula: 0.8-1.5 Na₂O. Al₂O₃. 0.8-6 SiO₂

These materials contain some bound water and are required to have a calcium ion exchange capacity of at least 50 mg CaO/g. The preferred sodium aluminosilicates contain 1.5-3.5 SiO2 units (in the formula above). Both the amorphous and the crystalline materials can be prepared readily by reaction between sodium silicate and sodium aluminate, as amply described in the literature. Suitable crystalline sodium aluminosilicate ion-exchange detergency builders are described, for example, in GB 1 429 143 (Procter & Gamble). The preferred sodium aluminosilicates of this type are the well-known commercially available zeolites A and X, and mixtures thereof.

The zeolite may be the commercially available zeolite 4A now widely used in laundry detergent powders. However, according to a preferred embodiment of the invention, the zeolite builder incorporated in the compositions of the invention is maximum aluminium zeolite P (zeolite MAP) as described and claimed in EP 384 070A (Unilever). Zeolite MAP is defined as an alkali metal aluminosilicate of the zeolite P type having a silicon to aluminium weight ratio not exceeding 1.33, preferably within the range of from 0.90 to 1.33, and more preferably within the range of from 0.90 to 1.20.

Especially preferred is zeolite MAP having a silicon to aluminium weight ratio not exceeding 1.07, more preferably about 1.00. The calcium binding capacity of zeolite MAP is generally at least 150 mg CaO per g of anhydrous material.

Organic builders that may be present include polycarboxylate polymers such as polyacrylates, acrylic/maleic copolymers, and acrylic phosphinates; monomeric polycarboxylates such as citrates, gluconates, oxydisuccinates, glycerol mono-, di and trisuccinates, carboxymethyloxy succinates, carboxymethyloxymalonates, dipicolinates, hydroxyethyliminodiacetates, alkyl- and alkenylmalonates and succinates; and sulphonated fatty acid salts. This list is not intended to be exhaustive.

Especially preferred organic builders are citrates, suitably used in amounts of from 5 to 30 wt %, preferably from 10 to 25 wt %; and acrylic polymers, more especially acrylic/maleic copolymers, suitably used in amounts of from 0.5 to 15 wt %, preferably from 1 to 10 wt %.

Builders, both inorganic and organic, are preferably present in alkali metal salt, especially sodium salt, form.

Compositions according to the invention may also suitably contain a bleach system. Fabric washing compositions may desirably contain peroxy bleach compounds, for example, inorganic persalts or organic peroxyacids, capable of yielding hydrogen peroxide in aqueous solution.

Suitable peroxy bleach compounds include organic peroxides such as urea peroxide, and inorganic persalts such as the alkali metal perborates, percarbonates, perphosphates, persilicates and persulphates. Preferred inorganic persalts are sodium perborate monohydrate and tetrahydrate, and sodium percarbonate.

Especially preferred is sodium percarbonate having a protective coating against destabilisation by moisture. Sodium percarbonate having a protective coating comprising sodium metaborate and sodium silicate is disclosed in GB 2 123 044B (Kao).

The peroxy bleach compound is suitably present in an amount of from 0.1 to 35 wt %, preferably from 0.5 to 25 wt %. The peroxy bleach compound may be used in conjunction with a bleach activator (bleach precursor) to improve bleaching action at low wash temperatures. The bleach precursor is suitably present in an amount of from 0.1 to 8 wt %, preferably from 0.5 to 5 wt %.

Preferred bleach precursors are peroxycarboxylic acid precursors, more especially peracetic acid precursors and pernoanoic acid precursors. Especially preferred bleach precursors suitable for use in the present invention are N,N,N′,N′,-tetracetyl ethylenediamine (TAED) and sodium nonanoyloxybenzene sulphonate (SNOBS). The novel quaternary ammonium and phosphonium bleach precursors disclosed in U.S. Pat. No. 4,751,015 and U.S. Pat. No. 4,818,426 (Lever Brothers Company) and EP 402 971A (Unilever), and the cationic bleach precursors disclosed in EP 284 292A and EP 303 520A (Kao) are also of interest.

The bleach system can be either supplemented with or replaced by a peroxyacid. Examples of such peracids can be found in U.S. Pat. No. 4,686,063 and U.S. Pat. No. 5,397,501 (Unilever). A preferred example is the imido peroxycarboxylic class of peracids described in EP A 325 288, EP A 349 940, DE 382 3172 and EP 325 289. A particularly preferred example is phthalimido peroxy caproic acid (PAP). Such peracids are suitably present at 0.1-12%, preferably 0.5-10%.

A bleach stabiliser (transition metal sequestrant) may also be present. Suitable bleach stabilisers include ethylenediamine tetra-acetate (EDTA), the polyphosphonates such as Dequest (Trade Mark) and non-phosphate stabilisers such as EDDS (ethylene diamine di-succinic acid). These bleach stabilisers are also useful for stain removal especially in products containing low levels of bleaching species or no bleaching species.

An especially preferred bleach system comprises a peroxy bleach compound (preferably sodium percarbonate optionally together with a bleach activator), and a transition metal bleach catalyst as described and claimed in EP 458 397A, EP 458 398A and EP 509 787A (Unilever).

The compositions according to the invention may also contain one or more enzyme(s).

Suitable enzymes include the proteases, amylases, cellulases, oxidases, peroxidases and lipases usable for incorporation in detergent compositions. Preferred proteolytic enzymes (proteases) are, catalytically active protein materials which degrade or alter protein types of stains when present as in fabric stains in a hydrolysis reaction. They may be of any suitable origin, such as vegetable, animal, bacterial or yeast origin.

Proteolytic enzymes or proteases of various qualities and origins and having activity in various pH ranges of from 4-12 are available and can be used in the instant invention. Examples of suitable proteolytic enzymes are the subtilisins which are obtained from particular strains of B. Subtilis B. licheniformis, such as the commercially available subtilisins Maxatase (Trade Mark), as supplied by Genencor International N.V., Delft, Holland, and Alcalase (Trade Mark), as supplied by Novozymes Industri A/S, Copenhagen, Denmark.

Particularly suitable is a protease obtained from a strain of Bacillus having maximum activity throughout the pH range of 8-12, being commercially available, e.g. from Novozymes Industri A/S under the registered trade-names Esperase (Trade Mark) and Savinase (Trade-Mark). The preparation of these and analogous enzymes is described in GB 1 243 785. Other commercial proteases are Kazusase (Trade Mark obtainable from Showa-Denko of Japan), Optimase (Trade Mark from Miles Kali-Chemie, Hannover, West Germany), and Superase (Trade Mark obtainable from Pfizer of U.S.A.).

Detergency enzymes are commonly employed in granular form in amounts of from about 0.1 to about 3.0 wt %. However, any suitable physical form of enzyme may be used.

The compositions of the invention may contain alkali metal, preferably sodium carbonate, in order to increase detergency and ease processing. Sodium carbonate may suitably be present in amounts ranging from 1 to 60 wt %, preferably from 2 to 40 wt %. However, compositions containing little or no sodium carbonate are also within the scope of the invention.

Powder flow may be improved by the incorporation of a small amount of a powder structurant, for example, a fatty acid (or fatty acid soap), a sugar, an acrylate or acrylate/maleate copolymer, or sodium silicate. One preferred powder structurant is fatty acid soap, suitably present in an amount of from 1 to 5 wt %.

Other materials that may be present in detergent compositions of the invention include sodium silicate; antiredeposition agents such as cellulosic polymers; soil release polymers; inorganic salts such as sodium sulphate; or lather boosters as appropriate; proteolytic and lipolytic enzymes; dyes; coloured speckles; fluorescers and decoupling polymers. This list is not intended to be exhaustive.

The detergent composition when diluted in the wash liquor (during a typical wash cycle) will typically give a pH of the wash liquor from 7 to 10.5 for a main wash detergent.

Particulate detergent compositions are suitably prepared by spray-drying a slurry of compatible heat-insensitive ingredients, and then spraying on or post-dosing those ingredients unsuitable for processing via the slurry. The skilled detergent formulator will have no difficulty in deciding which ingredients should be included in the slurry and which should not.

Particulate detergent compositions of the invention preferably have a bulk density of at least 400 g/litre, more preferably at least 500 g/litre. Especially preferred compositions have bulk densities of at least 650 g/litre, more preferably at least 700 g/litre.

Such powders may be prepared either by post-tower densification of spray-dried powder, or by wholly non-tower methods such as dry mixing and granulation; in both cases a high-speed mixer/granulator may advantageously be used. Processes using high-speed mixer/granulators are disclosed, for example, in EP 340 013A, EP 367 339A, EP 390 251A and EP 420 317A (Unilever).

Liquid detergent compositions can be prepared by admixing the essential and optional ingredients thereof in any desired order to provide compositions containing components in the requisite concentrations. Liquid compositions according to the present invention can also be in compact form which means it will contain a lower level of water compared to a conventional liquid detergent.

Product Forms

The composition of the invention may be in the form of a liquid, solid (e.g. powder or tablet), a gel or paste, spray, stick, bar or a foam or mousse. Examples include a soaking product, a rinse treatment (e.g. conditioner or finisher) or a main-wash product. Compositions suitable for direct application are preferred, such as gel or paste, spray, stick, bar, foam or mousse. The means for manufacturing any of the product forms are well known in the art. If the polymer particles are to be incorporated in a powder (optionally the powder to be tableted), and whether or not pre-emulsified, they are optionally included in a separate granular component.

Substrate

When used in laundering, the substrate may be any substrate onto which it is desirable to deposit polymer particles and which is subjected to treatment such as a washing or rinsing process.

In particular, the substrate may be a textile fabric.

Treatment

The treatment of the substrate with the material of the invention can be made by any suitable method such as washing, soaking or rinsing of the substrate but preferably by direct application such as spraying, rubbing, spotting, smearing, etc.

The treatment may involve contacting the substrate with an aqueous medium comprising the material of the invention.

The treatment may be provided as a spray composition e.g., for domestic (or industrial) application to fabric in a treatment separate from a conventional domestic laundering process. Suitable spray dispensing devices are disclosed in WO 96/15310 (Procter & Gamble) and are incorporated herein by reference. Alternatively, the composition may be applied through the irons water tank, a separate reservoir or a spray cartridge in an iron, as described in EP 1201816 and WO 99/27176.

### EXAMPLES

Embodiments of the present invention will now be explained in more detail by reference to the following non-limiting examples:

In the following examples where percentages are mentioned, this is to be understood as percentage by weight unless otherwise stated.

### Example 1

### Preparation of a Model Perfume

A simple model perfume was prepared and used in the ensuing examples. The model perfume components were selected on the following bases:
1) the component formed middle or base notes, and
2) the component was readily commercially available.

The components thus selected are shown in Table 1.

TABLE 1
Perfume Components used to make a simple model perfume.

| | Aldrich | | | |
|---|---|---|---|---|
| | Catalogue | | | b.p. |
| Component | Code | Formula | F.W. | (° C.) |
| α,α-Dimethyl phenethyl | W239208 | C₁₂H₁₆O₂ | 192 | 250 |
| acetate (DMPEA) | | | | |
| Methyl Dihydro | W340804 | C₁₃H₂₂O₃ | 226 | 110 at |
| Jasmonate (MDHJ) | | | | 0.2 mmHg |
| Phenethyl Phenylacetate | W286605 | C₁₆H₁₆O₂ | 325 | 325 |
| (PEPA) | | | | |

The composition of the model perfume is shown in Table 2.

TABLE 2
Composition of the simple model perfume.

| Component | Mass (g) | wt % |
|---|---|---|
| α,α-Dimethyl phenethyl | 7.8644 | 33.18 |
| acetate (DMPEA) | | |
| Methyl Dihydro Jasmonate | 7.9347 | 33.26 |
| (MDHJ) | | |
| Phenethyl Phenylacetate | 7.8445 | 33.55 |
| (PEPA) | | |

### Example 2

### Preparation of Polymer/Perfume Particles with LBG Deposition Aid by Hybrid Miniemulsion Polymerisation

Particles according to the invention were prepared by the hybrid miniemulsion polymerisation route in the presence of LBG as follows:

First, stock solutions were prepared as follows:
1. A 1% locust bean gum (LBG) stock solution was prepared by slowly dissolving LBG (5 g) in hot water (500 g) with continuous stirring. (This stock solution was kept refrigerated once made.)
2. A monomer/model perfume solution was prepared by dissolving model perfume (5 g) in butyl methacrylate (50 g) and then adding hexadecane (2 g).

Polymer/model perfume hybrid miniemulsion latex particles were then prepared using the stock solutions and the following method:
1. Sodium dodecyl sulphate (SDS, 0.25 g), Synperonic A20 (lg) and 50 g of 1% LBG stock solution was dissolved in demineralised water (358.75 g).
2. To the above mixture 57 g of the monomer/model perfume solution was added with mixing with an Ultratorax mixer at 20,000 rpm for 5 minutes, resulting in a crude emulsion.
3. Using a sonic probe the crude emulsion was then ultrasonicated at full power for 10 minutes, with stirring provided by a stirrer hot plate and stirrer flea in the crude emulsion. This resulted in the formation of miniemulsion droplets.
4. The miniemulsion droplets were placed in a suitable vessel, such as a polymerisation kettle, equipped with an over head stirrer, condenser, temperature controlled oil bath and a thermo-couple and heated until the mixture reached 75° C.
5. Once the mixture reached the desired temperature the initiation system was added, namely sodium bicarbonate (0.5 g) in water (10 g) plus ammonium persulphate (0.5 g) in water (10 g).
6. The polymerisation was allowed to proceed for 2 hours.
7. Once the reaction had finished, the reaction mixture was allowed to cool down and extra surfactant (Tween 80 (2 g) in water (10 g)) was added to help stabilise the latex.

The solids were determined gravimetrically by drying a known amount in an oven at 50° C. for 1 hour. They were found to be 10% of which 1% was perfume.

### Comparative Example A

A comparative example (A) containing no polymer particles, but 1% model perfume (emulsified perfume) was prepared, by adding surfactant stock solution (50 g) to demineralised water (445 g) and adding 5 g of model perfume. The mixture was shaken by hand to disperse and solubilise the perfume.

### Comparative Example B

A comparative example (B) was prepared using the method above, except the 1% LBG solution (50 g) was omitted and an additional 50 g of demineralised water was added at stage 1.

The solids were determined gravimetrically by drying a known amount in an oven at 50° C. for 1 hour. They were found to be 10% of which 1% was perfume.

### Example 3

### Preparation of Polymer/Perfume Latex Core Particles by Hybrid Miniemulsion Polymerization with PVAc Shells and LBG Deposition Aid

Particles according to the invention were prepared by the hybrid miniemulsion route to synthesis polymer/perfume core and then an additional LBG grafted PVAc shell was added via emulsion polymerisation as follows:

First, stock solutions were prepared as follows:
1. A 1% locust bean gum (LBG) stock solution was prepared by slowly dissolving LBG (5 g) in hot water (500 g) with continuous stirring. (This stock solution was kept refrigerated once made.)
2. A monomer/model perfume solution was prepared by dissolving model perfume (5 g) in butyl methacrylate (50 g) and then adding hexadecane (2 g).

Polymer/model perfume hybrid miniemulsion latex particles were then prepared using the stock solutions and the following method:
1. Sodium dodecyl sulphate (SDS, 0.25 g), Synperonic A20 (1 g) was dissolved in demineralised water (322.8 g).
2. To the above mixture 57 g of the monomer/model perfume solution was added with mixing with an Ultratorax mixer at 20,000 rpm for 5 minutes, resulting in a crude emulsion.
3. Using a sonic probe the crude emulsion was then ultrasonicated at full power for 10 minutes, with stirring provided by a stirrer hot plate and stirrer flea in the crude emulsion. This resulted in the formation of miniemulsion droplets.
4. The miniemulsion droplets were placed in a suitable vessel, such as a polymerisation kettle, equipped with an over head stirrer, condenser, temperature controlled oil bath and a thermo-couple and heated until the mixture reached 75° C.
5. Once the mixture reached the desired temperature the initiation system was added, namely sodium bicarbonate (0.5 g) in water (10 g) plus ammonium persulphate (0.5 g) in water (10 g).
6. The polymerisation was allowed to proceed for 2 hours.
7. Then vinyl acetate (25 g) and 1% LBG solution (50 g) were added to the reaction flask.
8. An initiator system of 35 wt % hydrogen peroxide (0.77 g) in water (5 g) and ascorbic acid (0.25 g) in water (5 g) was added.
9. Polymerisation was allowed to proceed for 1 hour.
10. Once the reaction had finished, the reaction mixture was allowed to cool down and extra surfactant (Tween 80 (2 g) in water (10 g)) was added to help stabilise the latex.

The solids were determined gravimetrically by drying a known amount in an oven at 50° C. for 1 hour. They were found to be 15% of which 1% was perfume.

### Comparative Example C

A comparative example (C) was prepared using the method above, except the 1% LBG solution (50 g) was omitted and an additional 50 g of demineralised water was added at stage 1.

The solids were determined gravimetrically by drying a known amount in an oven at 50° C. for 1 hour. They were found to be 15% of which 1% was perfume.

### Example 4

### Preparation of Polymer/Perfume Latex Core Particles by Artificial Miniemulsion with PVAc Shells and LBG Deposition Aid

Particles according to the invention were prepared by the artificial miniemulsion route as follows:

First, a solution of polymer/model perfume in dichloromethane (DCM) was prepared by dissolving model perfume (5 g) in poly butyl methacrylate (50 g) and hexadecane (2 g) in dichloromethane (DCM, 200 g).

Polymer/model perfume artificial miniemulsion latex particles were then prepared using the following method:
1. Sodium dodecyl sulphate (0.25 g), Synperonic A20 (1 g) were dissolved in demineralised water (343.8 g).
2. The polymer/model perfume/DCM solution (257 g) was added to the surfactant/water mixture with mixing with an Ultratorax mixer at 20,000 rpm for 5 minutes to form a crude emulsion.
3. Using a sonic probe, the crude emulsion was ultrasonicated at full power for 10 minutes with stirring provided by a stirrer hot plate and stirrer flea in the crude emulsion to form miniemulsion droplets.
4. The miniemulsion droplets were then placed in a suitable round bottom flask and the DCM removed using a rotary evaporator and gravimetric analysis to monitor the loss of DCM.
5. The miniemulsion droplets were placed in a suitable vessel, such as a polymerisation kettle, equipped with an over head stirrer, condenser, temperature controlled oil bath and a thermo-couple.
6. Vinyl acetate (25 g) and 1% LBG solution (50 g) were added to the reaction flask.
7. The reaction flask was heated until the mixture reached 75° C.
8. An initiator system of 35 wt % hydrogen peroxide (0.77 g) in water (5 g) and ascorbic acid (0.25 g) in water (5 g) was added.
9. Polymerisation was allowed to proceed for 1 hour.
10. Once the reaction had finished, the reaction mixture was allowed to cool down and extra surfactant (Tween 80 (2 g) in water (10 g)) was added to help stabilise the latex.

The solids were determined gravimetrically by drying a known amount in an oven at 50° C. for 1 hour. They were found to be 15% of which 1% was perfume.

### Comparative Example D

A comparative example (D) was prepared using the method above, except the 1% LBG solution (50 g) was omitted and an additional 50 g of demineralised water was added at stage 1.

The solids were determined gravimetrically by drying a known amount in an oven at 50° C. for 1 hour. They were found to be 15% of which 1% was perfume

### Example 5

### Wash Deposition of Comparative Example A and B, Example 2, Comparative Example C, Example 3, Comparative Example D and Example 4 onto Woven Cotton

The deposition of the following samples onto woven cotton under a simulated wash process was conducted:

Comparative Example A=Emulsified Model Perfume

Comparative Example B=Polymer/Perfume Miniemulsion (ME) Polymerised

Example 2=LBG grafted Polymer/Perfume ME Polymerised

Comparative Example C=Polymer/Perfume ME Polymerised Core−PVAC Shell
Example 3=Polymer/Perfume ME Polymerised Core−LBG-PVAC Shell
Comparative Example D=Polymer/Perfume Artificial (Art) Core−PVAC Shell

Example 4=Polymer/Perfume Artificial (Art) Core−LBG-PVAc Shell

The level of perfume contained in each of the above samples was 1%.

The wash procedure was as follows:

The Simulated Wash Process:

Preparation of Stock Solutions:

Surfactant Stock: (10 g/L 50:50 LAS:A7) was prepared by dissolving Linear Alkyl Benzene Sulphonate (9.09 g LAS (55% Active)) and Synperonic A7 (5 g) in de-ionised water to a total of 1 litre.

Base Buffer Stock: (0.1 M) was prepared by dissolving Sodium Carbonate (7.5465 g) and Sodium Hydrogen Carbonate (2.4195 g) in de-ionised water to a total of 1 litre.

Preparation of the Wash Liquor:

Base Buffer Stock (12.5 ml) and surfactant stock (12.5 ml) were added to a 500 ml Linitest pot and 100 ml de-ionised water was added to produce a wash liquor buffered at pH 10.5 and containing 1 g/L surfactant (50:50 LAS:A7).

Simulated Wash:

0.3 g of the above samples (equates to 0.003 g of perfume and 30 ppm of perfume on wash liquor) were each added to the linitest pots containing wash liquor and agitated slightly to ensure mixing. (Washes were done in duplicate for each sample and results averaged).

Linitest Equipment and Procedure

A section of unfluoresced cotton measuring 20 cm by 20 cm was placed into each linitest pot containing the wash liquor and polymer particles and the pot was sealed.

The Linitest is a proprietary laboratory scale washing machine (Ex. Heraeus). The equipment is designed and built to comply with the requirements for international standard test specifications. It is used for small scale detergency and stain removal testing particularly when low liquor to cloth ratios are required.

There are various models of the Linitest commercially available. The model used in this case has a single rotation speed of 40 rpm. The carrier is capable of accommodating twelve 500 ml steel containers and can be operated at temperatures up to 100° C.

General Principles:

The Linitest comprises a 20 litre tank, control system and drive mechanism. Permanent thermostatically controlled tubular heating elements in the base of the tank heat the bath liquor to the required temperature. The stainless steel construction throughout ensures efficient heat transfer to the specimen containers that are mounted on a rotating horizontal carrier driven by a geared motor. The rotating movement of the carrier ‘throws’ the liquid from one end of the container to the other in a continuous action. This movement simulates the mechanical washing process and additional mechanical action can be obtained by using steel ball bearings or discs.

Wash Conditions:

The linitest pots were attached to the Linitester cradle and rotated 45 minutes at 40° C. to simulate the main wash. The cloths were then removed and wrung by hand.

Simulated Rinses:

The linitest pots were then thoroughly rinsed and the ‘wrung’ cloths returned to the pots and 125 ml of de-ionised water was added. The linitester bath water was drained and the pots attached to the cradle and rotated for 10 minutes at ambient temperature (−20° C.) to simulate a rinse procedure. The clothes were then removed and wrung by hand. This procedure was repeated a further two times to simulate the second and third rinse. The final rinsed cloths were hung up overnight to dry.

Quantification of Perfume on Dried Fabrics

Each cotton swatch was cut into quarters (10×10 cm, 1.5 g) and added to 15 ml of tetrahydrofuran (THF) and agitated (Roller Mixer SRT2, ex. Stuart Scientific) overnight (˜15 hrs) to extract the perfume from the cloth. (The analysis was done in duplicate and 2 swatches from each sample were assessed). The level of perfume extracted from the fabric into the THF solvent was determined using a gas chromatogram with mass spectrum detector (GC-MS) using selected ion monitoring (SIM). A standard containing 30 ppm of model perfume was ran at the start of the analysis prior to running all samples. The GC-MS settings follow:

Oven—Initial 50° C. Hold for 2 minutes.

Ramp—20° C./min. Final temperature 300° C.

Total Run Time 14.50 minutes.

Column—HP-5HS 30 m×0.25 mm−0.25 μm thickness

Injector—1 μl, Splitless, Solvent delay=3 mins.

MS—Selected Ion Monitoring (SIM)

The parameters used for the SIM analysis are shown in Table 3.

TABLE 3
SIM parameters for the MS to detect and quantify
each component in the simple model perfume.

| | RT | Ion | Time Window |
|---|---|---|---|
| Component | (mins) | m/e | (mins) |
| α,α-Dimethyl phenethyl | 8.21 | 132 | 3-9 |
| acetate (DMPEA) | | | |
| Methyl Dihydro Jasmonate | 10.34 | 156 | 9-11 |
| (MDHJ) | | | |
| Phenethyl Phenylacetate | 11.84 | 104 | 11-14.5 |
| (PEPA) | | | |

Calculation of Percentage Perfume Deposition

If all perfume was deposited (0.003/4=0.00075 g) and extracted from the quartered (10×10 cm) cloth into the 15 ml of THF the concentration detected would be 50 ppm. The ratio of the sum of the 3 peaks for each sample to the peak area sum of the standard (×30) gave a measure of the level (in ppm) of the perfume that was deposited on the cloth. The ratio of this value over the 50 ppm maximum level that could be present (×100) gave a measure of the percentage perfume deposited.

The percentage perfume deposited for Comparative Example A and B, Example 2, Comparative Example C, Example 3, Comparative Example D and Example 4 are shown in Table 4.

TABLE 4
Comparison of Percentage deposited during a simulated wash process
for Comparative Example A and B, Example 2, Comparative Example
C, Example 3, Comparative Example D and Example 4

| | | % Wash |
|---|---|---|
| Sample | Details | Deposition |
| Comparative | Emulsified Model Perfume | 1.6 |
| Example A | | |
| Comparative | Polymer/Perfume Miniemulsion | 4.1 |
| Example B | Polymerised | |
| Example 2 | LBG grafted Polymer/Perfume | 5.5 |
| | Miniemulsion Polymerised | |
| Comparative | Polymer/Perfume Miniemulsion | 1.4 |
| Example C | Polymerised Core - PVAc Shell | |
| Example 3 | Polymer/Perfume Miniemulsion | 7.3 |
| | Polymerised Core - LBG-PVAc | |
| | Shell | |
| Comparative | Polymer/Perfume Artificial Core - | 0.9 |
| Example D | PVAc Shell | |
| Example 4 | Polymer/Perfume Artificial Core - | 18.9 |
| | LBG-PVAc Shell | |

The results show the significant enhancement of deposition achieved from the LBG grafted variants compared to that of emulsified perfume.

The results further show that perfume deposition can be enhanced using particles with a core comprising perfume and a shell; with the deposition aid grafted to the shell.

1. A polymer particle comprising a core wherein the core comprises a perfume and a polymer comprising monomer units which are derived from monomers selected from the group consisting of:
a) monomers with a solubility in water of less than 0.1 g/l, and/or
b) monomers with a solubility in water of from 0.1 to 30 g/l, and
c) optionally, monomers with a solubility in water of greater than 30 g/l, and/or
d) optionally, cross linkers,
wherein the particle further comprises a shell, wherein the shell comprises monomer units selected from b), and optionally c) and/or optionally d)
and wherein the particle further comprises a nonionic deposition aid.

2. A particle as claimed in claim 1, wherein the deposition aid is a polysaccharide having β-1,4 linkages.

3. A particle as claimed in claim 2, wherein the polysaccharide is selected from the group consisting of a polymannan, a polyglucan, a polyglucomannan, a polyxyloglucan and a polygalactomannan or a mixture thereof.

4. A particle as claimed in claim 3, wherein the polysaccharide is locust bean gum.

5. (canceled)

6. A particle as claimed in claim 1 wherein the perfume is present in an amount of from 1 to 50% by total weight of the particle, preferably from 10 to 50% by total weight of the particle.

7. A particle as claimed in claim 1 wherein the perfume has a molecular weight of from 50 to 500.

8. A particle as claimed in claim 1 wherein the perfume has a boiling point of from 30 to 500 degrees Centigrade.

9. A particle as claimed in claim 1 wherein the polymer comprises monomer units which are derived from monomers selected from the group consisting of olefins, ethylene, vinylaromatic monomers, esters of vinyl alcohol with mono- and di-carboxylic acids, esters of α,β-monoethylenically unsaturated mono- and dicarboxylic acids with alcohols, nitriles of α,β-monoethylenically unsaturated carboxylic acids, conjugated dienes, α,β-monoethylenically unsaturated monocarboxylic and dicarboxylic acids and their amides, methacrylic acid and its esters with alcohols and diols, acrylic acid and its esters with alcohols and diols, dimethyl or di-n-butyl maleate, and/or vinyl-sulfonic acid and its water-soluble salts and mixtures thereof.

10. A particle as claimed in claim 1 wherein the monomers a), b), c) and d) comprise the following:
a) vinyl octate; Vinyl decanote, vinyl laurate; vinyl stearate; esters of acrylic, methacrylic, maleic, fumaric or itaconic acid with decyl, dodecyl, tetadecyl, hexadecyl and octadecyl alcohol,
b) styrene; α-methylstyrene; o-chlorostyrene; vinyl acetate; vinyl propionate;
vinyl n-butyrate; esters of acrylic, methacrylic, maleic, fumaric or itaconic acid with methyl, ethyl, n-butyl, isobutyl, n-hexyl and 2-ethylhexyl alcohol; 1,3-butadiene; 2,3 dimethyl butadiene; and isoprene,
c) acrylic acid, methacrylic acid, maleic acid, fumaric acid, itaconic acid, poly (alkylene oxide) monoacrylates and monomethacrylates, N-vinyl-pyrrolidone, methacrylic and acrylic acid, 2-hydroxyethyl acrylates and methacrylates, glycerol acrylates and methacrylates, poly(ethylene glycol) methacrylates and acrylates, n-vinyl pyrrolidone, acryloyl morpholine, vinyl formamide, n-vinyl acetamide and vinyl caprolactone, acrylonitrile, acrylamide, and methacrylamide at levels of less than 10% by weight of the monomer unit content of the particle; 2-(dimethylamino) ethyl methacrylate, 2-(diethylamino) ethyl methacrylate, 2-(tert-butylamino) ethyl methacrylate, 2-aminoethyl methacrylate, 2-(2-oxo-1-imidazolidinyl) ethyl methacrylate, vinyl pyridine, vinyl carbazole, vinyl imidazole, vinyl aniline, and their cationic forms after treatment with alkyl halides,
d) vinyltoluenes, divinyl benzene, ethylene glycol diacrylate, 1,2-propylene glycol diacrylate, 1,3-propylene glycol diacrylate, 1,3-butylene glycol diacrylate, 1,4-butylene glycol diacrylates, ethylene glycol dimethacrylate, 1,2-propylene glycol dimethacrylate, 1,3-propylene glycol dimethacrylate, 1,3-butylene glycol dimethacrylate, 1,4-butylene glycol dimethacrylate, divinylbenzene, vinyl methacrylate, vinyl acrylate, allyl methacrylate, allyl acrylate, diallyl maleate, diallyl fumarate, methylenebisacrylamide, cyclopentadienyl acrylate, and triallyl cyanurate.

11. A particle as claimed in claim 1 wherein the polymer comprises monomer units, which are derived from monomers selected from the group consisting of butyl acrylate, butyl methacrylate, hexyl acrylate, hexyl methacrylate, 2-ethylhexyl acrylate, 2-ethylhexyl methacrylate, dodecyl acrylate, dodecyl methacrylate, octadecyl acrylate, octadecyl methacrylate, styrene, vinyl acetate and divinyl benzene, or mixtures thereof.

12. A particle as claimed in claim 1, having a particle size of less than 1 micron, preferably of less than 500 nm.

13. A process for the preparation of polymer particles as claimed in claim 1, which comprises preparation of a miniemulsion.

14. A process as claimed in claim 12 which comprises the steps of:
(a) preparation of a miniemulsion (comprising monomers, a perfume and a cosurfactant), and
(b) polymerisation of the miniemulsion of step (a) to form miniemulsion core particles, and
(c) attachment of a deposition aid around the core particles of step (b).

15. A process as claimed in claim 12 which comprises the steps of:
(a) preparation of a miniemulsion (comprising monomers, a perfume and a cosurfactant), and
(b) polymerisation of the miniemulsion of step (a) to form miniemulsion core particles, and
(c) addition of shell monomers and initiator such that polymerisation occurs to form a shell around the core particles of step (b), and attachment of a deposition aid around the particles.

16. A process as claimed in claim 13 which comprises the steps of:
(i) mixing monomers with a cosurfactant to form a mixture (y),
(ii) dissolving at least one surfactant in water to form a mixture (z),
(iii) combining (y) and (z) and subjecting to high shear to form a miniemulsion,
(iv) adding an initiator such that polymerisation proceeds to form a core, and
(v) adding monomers, a deposition aid and initiators at such a rate so as to polymerise the monomers resulting in a shell around the core, with simultaneous attachment of a deposition aid onto the particles.

17. A process as claimed in claim 14 which comprises the steps of:
(a) preparation of a miniemulsion (comprising a polymer, a perfume, a cosurfactant and a water insoluble volatile solvent),
(b) removal of the volatile solvent, and
(c) addition of shell monomers and initiator such that polymerisation occurs to form a shell around the core particles of step (b),
wherein attachment of a deposition aid to the particles occurs in step (c).

18. A process as claimed in claim 16 which comprises the steps of:
(i) dissolving polymer and a perfume with a cosurfactant in a water insoluble volatile solvent to form a mixture (m),
(ii) dissolving at least one surfactant in water to form a mixture (n),
(iii) combining (m) and (n) and subjecting to high shear to form a miniemulsion,
(iv) removing the volatile solvent, and
(v) adding shell monomers, a deposition aid and initiators at such a rate so as to polymerise the monomers resulting in a shell around the core, with simultaneous attachment of a deposition aid onto the polymer particles.

19. A laundry treatment composition comprising the particle as claimed in claim 1.

20. Use of a laundry treatment composition as claimed in claim 18 in the treatment of fabric.

21. Use as claimed in claim 19 wherein the use provides a deposition benefit for the perfume onto the fabric.

22. A method of treating fabric, comprising contacting the fabric with the polymer particle according to claim 1.

23. Use of a particle as claimed in claim 1 in the treatment of a fabric to provide a perfume deposition benefit during a laundry process.

24. Use of a particle as claimed in claim 1 in the manufacture of a laundry treatment composition to provide a perfume deposition benefit during a laundry process.

25. Aqueous wash medium comprising from 0.05 to 1 gram per litre of a particle according to claim 1.
